(19) United States
(12) Patent Application Publication
(10) Pub. No.: US 2021/0015427 A1
(43) Pub. Date: Jan. 21, 2021

(54) EASILY CUSTOMIZABLE MULTI-SHELL MEG HELMET

(71) Applicant: QuSpin, Inc., Louiville, CO (US)

(72) Inventors: Vishal Shah, Superior, CO (US); Shao Bo Zhou, Denver, CO (US); Christian Fahrenbruck, Westminster, CO (US)

(73) Assignee: QuSpin Inc., Louisville, CO (US)

(21) Appl. No.: 16/517,540

(22) Filed: Jul. 20, 2019

(51) Int. Cl. A61B 5/00; A61B 5/04

(52) U.S. Cl. CPC A61B 5/6803; A61B 5/0478; A61B 5/04008

(57) ABSTRACT

A wearable and customizable multi-shell MEG helmet comprising an inner shell and outer shell, wherein the inner shell interior surface is customized to conform to the patient's head shape so that the helmet assembly moves in unison with the patient's head movement and sensor locations are controlled and remain fixed relative to the brain. This invention improves data quality and user comfort since head movements may be permitted and their effects on data integrity is minimized. The outer shell is generic and may fit over any customized inner shell. The outer shell holds a group of sensors, which may be, but not limited to, optically pumped magnetometers. This generic outer shell may mate with the inner shell, allowing sensors to be easily pushed into the inner shell to be in closer proximity to the patient's head. Furthermore, this multi-shell MEG helmet design allows an easy and convenient way to transfer sensors from one patient to the next patient because the need to remove and reinstall individual sensors is avoided. The helmet may contain cable and other connector means that provides the electrical connections for communication with and control of individual sensors.

The following application is an application for patent under 35 USC 111 (a). This invention was made with government support under R44 MH110288 awarded by National Institutes of Health (NIH). The government has certain rights in the invention.

### FIELD OF INVENTION

This disclosure relates to the field of magnetoencephalography and a device for measuring magnetic fields produced by electrical currents in the brain and method of manufacture thereof.

### BACKGROUND

Magnetoencephalography (MEG) is a functional neuroimaging technique for mapping brain activity by recording magnetic fields produced by electrical currents occurring naturally in the brain using very sensitive magnetometers. Arrays of SQUID (superconducting quantum interference device) detectors are the most common magnetic field sensors used in MEG. Because the SQUID sensors must be cooled to cryogenic temperatures, the helmet or device for measuring brain waves in a SQUID based MEG scanner is in many times large and may not conform to the user's head shape.

The one-size fits all helmet in SQUID-MEG, as shown in prior art FIG. 1A, can lead to substantial loss in signal fidelity for many patients, especially children. In addition, the fixed helmet in SQUID-MEG systems, such as in the prior art FIG. 1B restricts head motion making it difficult to utilize SQUID-MEG in more naturalistic usage scenarios.

Recently a different type of magnetometer, Optically Pumped Magnetometers (OPMs) with sensitivity similar to SQUID sensors have become commercially available. The OPMs, also called atomic magnetometers, optical magnetometers, or optical atomic magnetometers, operate by optically measuring the spin dynamics of alkali or noble gas atoms to infer external magnetic field with very high sensitivity.

### SUMMARY OF THE INVENTION

These OPMs are compact, lightweight, and do not require cryogenic cooling. Consequently, with OPMs it is now possible to develop advanced MEG helmets that improve patient comfort and system performance by allowing the sensors to be placed closer to the brain resulting in higher accuracy scans. The present invention is an easily customizable multi-shell MEG helmet that utilizes noncryogenic optically pumped magnetometers (OPMs) with advantages of precisely controlled sensor location relative to the brain, convenience for the user, increased comfort for the patient, and reduced subject preparation time due to ease of fitting onto and removing the helmet from a patient.

Typically, MEG brain scanner systems, like those shown in the prior art FIG. 1A, utilize a number of highly sensitive magnetic field or gradient magnetic field sensors that can be placed inside a helmet device which operates as a scanner. The electrical firing of the neurons inside a brain generates magnetic fields which passes through the brain tissue and the skull largely unhindered. The sensors inside the MEG helmet, which may be OPMs, can non-invasively detect and localize this electrical activity with high accuracy using advanced forms of triangulation with a large number of sensors in the helmet.

To accurately localize and pinpoint the source of electrical activity inside the brain, it is important to know the location of the magnetic field sensors in the scanner with respect to the head and the brain. In a SQUID-MEG scanner which has a fixed helmet or cage around the head as in prior art FIG. 1A, only the positions of the sensors inside the helmet or cage are known. To localize the position and orientation of the head with respect to the helmet, the patient wears small active magnetic coils over the head at known locations. Using the signatures from the active coils, the position and location of the head inside the helmet is mathematically estimated.

Brain cells (neurons) communicate with each other by generating tiny electrical currents. The flow of electrical current produces a magnetic field, which can then be recorded using sensitive magnetic sensors. Because the strength of the magnetic field produced by the brain is so small, very specialized instrumentation is required to pick up the signal.

Traditionally, these sensing systems consisted of small, high-resolution coils, coupled to devices called SQUIDs (superconducting quantum interference devices). More than 300 of these specialized sensors can be arrayed inside a head cavity providing whole-head coverage with high resolution capabilities as shown in FIG. 1A. By analyzing the patterns of the signals recorded by all of these sensors, the location, strength and orientation of the sources can be inferred.

Unlike the SQUID sensors, the OPMs are light weight and flexible and can be placed anywhere on a helmet to improve signal quality. OPMs are passive magnetic field sensors that operate by optically measuring the spin dynamics of spin polarized alkali or noble gas atoms. They have three main components including a laser, a vapor cell containing ‘sensing’ atoms in a gaseous state, and a photodetector.

University College London and Nottingham university (UCL/Nott) have developed a 3D printed helmet built to match the head shape of an individual patient as published at www.nature.com/articles/nature26147 and shown in prior art FIG. 1B. Each helmet is precisely shaped to fit a particular patient. The UCL/Nott helmet has slots to hold the OPM sensors at predefined locations. Thus, when the subject wears the custom helmet, the position of the OPM sensors is accurately known with respect to the brain, largely without the need for additional software based mathematical estimations. While the UCL/Nott solves the sensor-brain positioning problem, the use of the prior art helmet shown in FIG. 1B in a clinical or hospital setting could be cumbersome and time consuming due to the need for the sensors to be moved from one helmet to the next each time a new patient is scanned.

As shown in FIG. 2, the present invention is an easily customizable two-shell MEG helmet which has the advantages of precisely controlled sensor location relative to the brain with greatly reduced helmet preparation time. The present invention comprises a multi- or two-shell helmet in which a rigid or semi-rigid inner shell is 3D printed or additively manufactured such that its interior surface conforms to or matches the exact head shape of the patient. In addition, the inner shell includes slots or openings extending from the interior surface to exterior surface of the inner shell to position OPMs or other sensors over the head. The exterior surface of the inner shell may be designed to match sensor positions on an outer shell. A flexible or semi-flexible outer shell of the helmet houses the sensors and is generic in that it can be fitted over any inner shell and provides supporting structure to hold all the sensors and cords or cables, as a unified matrix.

With this two-shell design, helmet preparation time is greatly minimized by allowing the inner shell to be easily replaced and mated to the outer shell containing the sensor matrix without the need to individually replace sensors with each patient. The interior surface of the inner shell is customized to fit the precise shape and form of each patient's head and may be a rigid or semi-rigid in form, while the outer shell may be flexible, semi-rigid or rigid, fitting over the inner shell and used for a patient with any head size or shape.

The inner shell may be fabricated by first taking a 3D image or producing a model of the patient's head. An image or 3D model can be produced by optical/MRI/x-ray scanning, or other suitable method for obtaining 3D spatial data, or creating a mold and then creating a model from said mold. This data and/or model may be used to create the interior surface shape of the inner shell for instance using computer aided design, then 3D printing it or using another additive manufacturing device or other means of producing a 3D structure. Additive manufacturing, including 3D printing, refers to several technologies that produce parts in an additive way. The starting point is a digital 3D model of a part, an inner shell in this case, which is then “sliced” in thin layers by a specific computer software. An additive manufacturing machine or 3D printer builds these layers on top of another and thus creates the physical part. Additive manufacturing or 3D printing devices may use starting materials such as ceramics, metals, sand, plastics, waxes, and/or other starting materials to create devices with a 3D structure that may be in the case of the inner shell rigid, semi-rigid, or semi-flexible in form. Technologies for 3D printing may include but not be limited to binder jetting, electron beam melting, fused deposition modeling, hybrid processes, laser melting, laser sintering, material jetting, photopolymer jetting, and stereolithography. The inner shell may be fabricated with one or more openings to receive sensors, for instance OPM sensors, and/or reduce weight or create ventilation for the head of the patient. It is conceived that technological advances may make it possible to develop a helmet not requiring these openings. Further, one or more attachment devices may be fitted to or integral with the inner shell for holding the shell in a defined position on the patient's head.

The outer shell or cap may be a flexible, semi-rigid, or a rigid cap. Various embodiments may be made for instance of cotton, synthetic fabric, plastic, vinyl, silicone, or natural or synthetic rubber, in a roughly round or oval shape of a hat or cap with openings for face and ears. It may be constructed in a size to be larger than most heads in order to mate with the inner shell of any size and shape.

Mating features between the inner and outer shell may be used to maintain the positions of the shells relative to each other and make it easier to mate the inner and outer shells. Head movements translate from the inner shell to the outer shell and by extension to all sensors in unison. The mating features can be used to achieve rigidity between inner and outer shells.

Initially, one or more sensors are housed on the outer shell inside holders. The sensors can be placed in these holders in a random or specific pattern. The number of the sensors can vary as well. In addition to OPM sensors, the helmet may also accommodate other sensors, such as electroencephalogram (EEG) electrodes, functional near infrared spectroscopy (fNIRS) sensors, accelerometers or gyroscopes for example. When the outer shell is fitted over the inner shell, the sensors are matched with openings in the inner shell and sensors slid into these openings so that the sensors may contact the skull of the patient.

Electronics in the form of cords or cables connect each sensor to the controller and software. The outer shell may incorporate features for routing sensor cabling. The outer shell, or multiple additional shells, may have the cabling for the sensors built-in and it may have connectors that allow the sensors to be plugged directly into the outer shell. The cables may be a combination of electrical wires, and/or flexible or rigid printed circuit boards.

The two-shell MEG helmet may be secured one shell at a time or the two shells may be mated prior to fitting over the patient's head. The two-shell MEG helmet may be held in place by a fastening mechanism which may include, but is not limited to, straps that can be adjusted for tightness for user comfort, buckles, snaps, and/or VELCRO or any other means of semi-permanent attachment and adjustment. The straps may be made for instance of fabric, plastics, vinyl, or natural or synthetic rubber.

Every assembled two-shell MEG helmet has an interior shell with interior surface conforming precisely to the patient's head. The inner shell can be mounted first over the head and then the outer shell is placed over the inner shell. Alternatively, the helmet can be first assembled with all components installed in place and then the complete helmet is fitted and strapped over the patient's head.

The sensors can be manually or with an actuation mechanism pushed through the outer and the inner shells such that the sensors are at the desired proximity to the brain. MEG scan, patient preparation time, sensor configuration, and procedures are greatly simplified by the two-shell MEG helmet disclosed in more detail in the following drawings and descriptions.

### BRIEF DESCRIPTION OF THE DRAWINGS

FIG. 1A is an illustration of a prior art device.

FIG. 1B is an illustration of another prior art device.

FIG. 2 is a perspective view of the two-shell MEG helmet of the present invention.

FIG. 3 is an exploded view of the components of the present invention.

FIG. 4 is an example illustration of a two-shell MEG helmet of the present invention with an inner shell and flexible outer shell.

FIG. 5 is an example illustration of an outer shell made from rigid material.

FIG. 6. is an illustration of the present invention on a patient.

FIG. 7 is an illustration of a cross-section of the present invention.

Before explaining the disclosed embodiments of the present invention in detail, it is to be understood that the invention is not limited in its application to the details of the particular arrangement shown, since the invention is capable of other embodiments. Also, the terminology used herein is for the purpose of description and not of limitation.

### DETAILED DESCRIPTION OF THE INVENTION

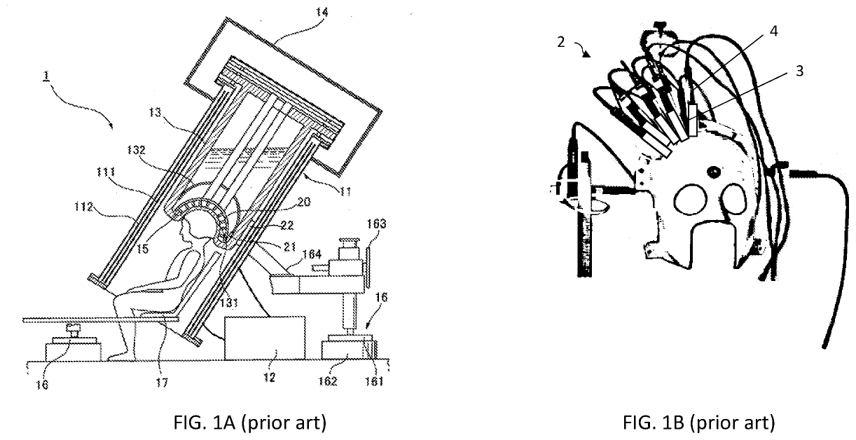

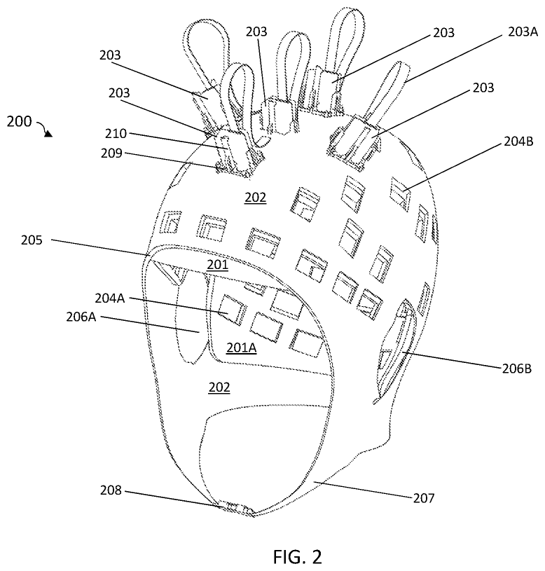

Illustrated in FIG. 1A is prior art SQUID MEG apparatus 1 for measuring the neuro-magnetic field from a human brain, being the invention of Matsui, et al. taken from U.S. Pub. No. 2005/0272996 A1 dated Dec. 5, 2008. The conventional SQUID or MEG apparatus 1 comprises a vacuum structure 11 of hollow cylinder for thermal insulation, a closed-cycle Helium refrigerator 12, a liquid-Helium dewar 13, and a top cover 14. The SQUID magnetic sensors 15 are fixed on a support block 20 around a head accommodating area 131. A first cylinder 111 of high critical temperature super-conductor material is cooled down to around the liquid nitrogen temperature and the outer cylinder 112 of high-permeability magnetic material are both arranged coaxially in its annular space. A superconducting canopy 132 is arranged above the magnetic sensors. A first 22 and second pillow 21 structure are placed to fill the gap between the superconducting canopy. The vacuum structure is set on the floor via four mechanical vibration-suppressor supports 16 each comprising a mechanical vibration-absorber 161 and an anti-mechanical vibration mechanism 162. The mechanical vibration-suppressor support 16 includes an up-and-down mechanism 163 which can be removed. The patient is seated on a non-magnetic chair 17.

Illustrated in FIG. 1B is the prior art UCL/Nott helmet 2 built to match the head shape of an individual patient as published at www.nature.com/articles/nature26147. The UCL/Nott helmet has slots 3 to hold the OPM or other sensors 4 at predefined locations as can be seen on top of the helmet.

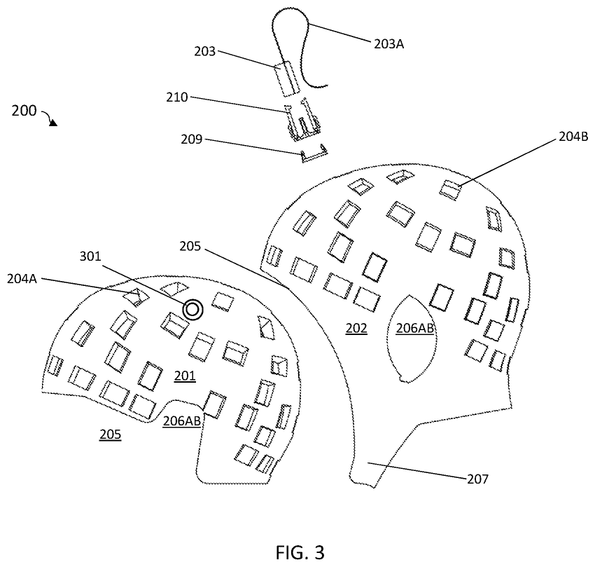

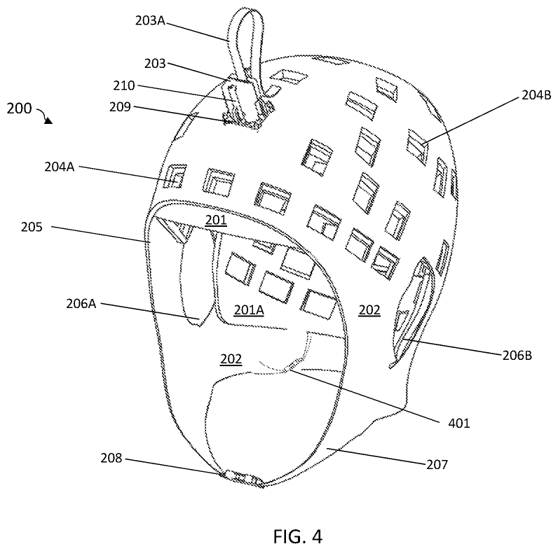

FIG. 2 is an illustration of the present invention, an easily customizable two-shell MEG helmet 200, comprising an inner shell 201 and outer shell 202. The inner shell 201 is rigid, semi-flexible, or semi-rigid and designed to precisely fit the shape of an individual patient and is described in further detail in FIG. 3. The interior surface 201A of the inner shell 201 is designed to conform to the shape of a particular patient's head. It is envisioned that this patient is a human but a shell designed for other animals or uses is contemplated. The outer shell 202 may be flexible, semi-rigid, or rigid and is described and shown further in FIGS. 3, 4, and 5. Sensors 203, being OPMs or other sensors, with sensor wires, cords, and/or cables 203A that connect the sensors to controllers and data systems, as described above. Use of other sensors such as electroencephalogram (EEG) electrodes, functional near infrared spectroscopy (fNIRS) sensors, accelerometers or gyroscopes for example is contemplated. As technology develops other sensors that may be used for MEG applications. These can be fitted in slots 204A, 204B arranged in desired patterns or random spots in both the inner 201 and outer shells 202. Openings for the face 205 and ears 206A, 206B may be provided along with a strap 207 on the outer shell 202 that may have a means of attachment 208, which may be optional, of one strap side to the other being buckle, snap, button, VELCRO, or any other means known for semi-permanently attaching one piece to another. Alternately the strap 207 may be one continuous piece. The buckle or other attachment means 208 may be placed at any point on the strap. A bracket 209 may be designed via 3D printing, described below, or other means of manufacture to fit openings for the sensors 204A,B. A sensor clip 210 may hold the sensors 203 in place and be designed to mate to the bracket 209 to hold the sensors in position on the two-shell MEG helmet 200.

FIG. 3 is an exploded view showing all of the components of the present invention including an inner shell 201 with interior surface (not shown) designed to conform to a patient's skull, outer shell 202, bracket 209, sensor clip 210, sensor 203, and sensor cable 203A. The inner shell 201 is a rigid or semi-rigid shell with an interior surface (not shown) designed to fit a particular patient's head. The head surface may be imaged using 3D scanning or MRI technology or a mold may be created to duplicate the outer shape of the head. An image or 3D model can be produced by optical scanning, or other suitable method for obtaining 3D spatial data, or creating a mold and then creating an image from said mold. This 3D image and/or model may be used to create a file or code sequence of computer language instructions using computer software programs, to fabricate the inner shell for instance using 3D printing or additive manufacturing or any device capable of producing a 3D structure. Additive manufacturing, or 3D printing, refers to several technologies that produce parts in an additive way. The starting point is a digital 3D model of a part, an inner shell 201 in this case, which is then “sliced” in thin layers by a specific computer software. An additive manufacturing machine or 3D printer builds these layer on top of another and thus creates the physical part. Additive manufacturing or 3D printing devices may use starting materials such as semi-flexible, semi-rigid, or semi-flexible ceramics, metals, sand, plastics, waxes, and/or other starting materials to create devices with a 3D structure. Technologies for 3D printing may include but not be limited to binder jetting, electron beam melting, fused deposition modeling, hybrid processes, laser melting, laser sintering, material jetting, photopolymer jetting, and stereolithography.

As shown in FIG. 3, the inner shell 201 may be fabricated with one or more openings 204A to receive sensors 203, and/or reduce weight or create ventilation for the head of the patient. The inner shell 201 may be fabricated to leave openings for the patient's face 205, ears 206A, B. Further, one or more attachment devices may be fitted to or integral with the inner shell for holding the shell in a defined position on the patient's head (not shown).

Also illustrated in FIG. 3, the outer shell 202 is larger than the inner shell 201 and is constructed to fit over the inner shell. It is not specific to any patient and is designed to fit over inner shells 201 of a range of sizes and shapes. It may be a flexible, semi-rigid, or a rigid cap or shell. Various embodiments may be made for instance of natural fibers such as cotton, or synthetic fabric, or blends of the two, or vinyl, plastic, or any suitable material in a roughly round or oval shape of a hat or cap with openings for face 205 and ears 206A, 206B. Additionally openings for sensors 204B as well as sensor holders 209, 210 are integral in the outer shell 202. It may be constructed in a size to be larger than most heads and a head fitted with the inner shell described above, in order to mate with a wide range of size and shape the inner shell may take. Two or more magnetometers or other sensing devices or an array of sensors 203 and their cords 203A, which may be OPM sensors or an array of sensors as described above, may be attached in random or defined patter to the outer cap for mating with the inner shell. Further one or more attachment devices (not shown) may be fitted to or integral with the outer cap strap 207. In addition, the inner and outer shell may have mating means affixed thereto for securing one shell to another such as a snap mating means 301 shown here with male side, female side would fit on the interior surface of the outer shell 202. Other semi-permanent means of attachment are envisioned including but not limited to hooks, buckles, belts, adhesive, and/or Velcro for securing one shell to the other.

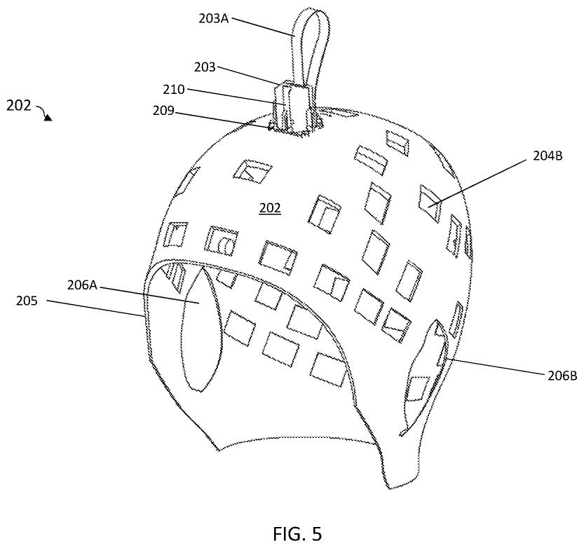

FIG. 4 illustrates an example embodiment of the two-shell MEG helmet 200 of the present invention wherein the inner shell 201 is a rigid or semi-rigid material with interior surface 201A designed to conform to the patient's head, and an outer shell 202 is fabricated from a more flexible material which may be fabric of natural or synthetic fibers with or without some elasticity, soft plastic, or vinyl. Any flexible material that can be fabricated with openings for the face 205 and ears 206A,B may be suitable manufacturing material. A strap 207 fitted with an attachment means 208, which may be optional, holds the outer shell 202 in place over the inner shell. Brackets 209 and sensor clips 210 may hold sensors 203 on the outer shell 202. In addition, the inner shell and outer shell may have mating means 401, such as a buckled strap shown here, or other means of semi-permanent attachment including hooks, snaps, Velcro, or the like, attached thereto for securing one shell to the other

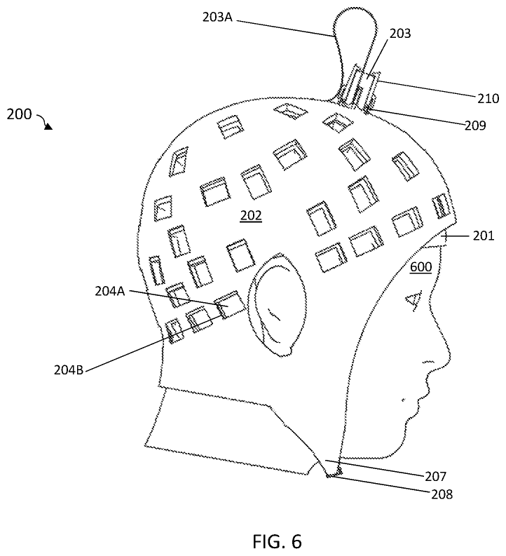

FIG. 5 illustrates an example of a second embodiment of the outer shell 202 wherein the shell is fabricated from a more rigid material. Materials described above including ceramics, metals, sand, plastics, vinyl, and/or waxes, either natural or synthetic may be suitable. Any semi-rigid or rigid material that can be fabricated with openings for the face 205 and ears 206A,B may be used as manufacturing material. Brackets 209 and sensor clips 210 may hold sensors 203 on the outer shell 202. Further one or more attachment devices may be fitted to or integral with the outer cap (not shown) to keep the helmet secured to the patient's head.

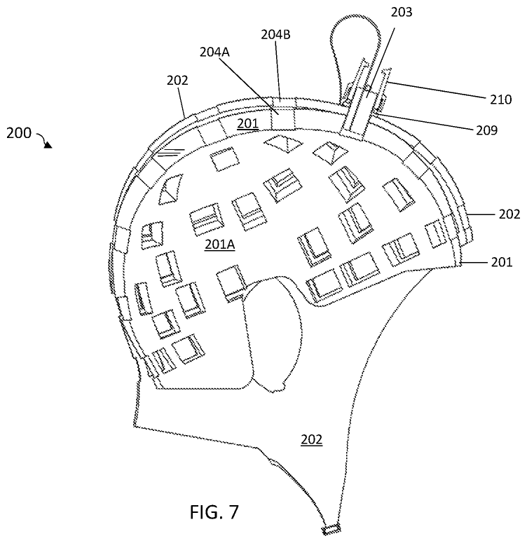

FIG. 6 illustrates the two-shell MEG helmet 200 as would be fitted on and used on a patient's head 600. In this illustration as in FIG. 4, the outer shell 202 is made of flexible material and includes a strap 207 and buckle 208 for affixing the helmet to the patient's head in a semi-permanent manner. In use, the patient's head is first modeled using 3D modeling or molds to create the inner shell 201 with interior surface (not shown) to conform to the shape of their skull. Once the inner shell 201 is manufactured it is fitted on the patient's head 600. The outer shell 202 with sensors 203, brackets 209, and sensor clips 210, is fitted atop the inner shell 201. The sensor openings 204A of the inner shell 201 are matched with the sensor openings 204B of the outer shell 202. The two-shell MEG helmet 200 is secured to the head using a fastening mechanism such as one or more straps 207 and buckle 208 or other attachment device (not shown). Because the inner shell conforms closely to the form and shape of the patient's head 600, the two-shell MEG helmet 200 moves with the patient and maintains the sensors position relative to the head. Sensor cables 601 connect electronics of the sensor with electronics for data collection and sensor control.

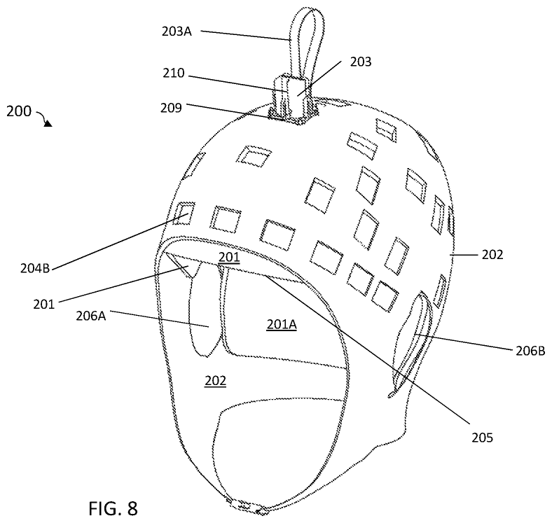

FIG. 7 is a cross-sectional view of the two-shell MEG helmet 200 with rigid inner shell 201, interior surface 201A designed to fit a particular patient's head shape, and flexible outer shell 202. The sensor bracket 209 and clip 210 integrated with the outer shell 202 house the sensor(s) 203. The outer shell 202 is fitted over the inner shell 201 and inner 204A and outer 204B shell openings are aligned. The sensor then can be guided into the sensor opening 204 B in the outer shell 202 and into the sensor opening 204A of the inner shell 201. This cross-sectional view illustrates that the features on the outer shell 202 for holding the sensor is aligned with the features on the inner shell 201, thus allowing the sensor to be pushed in manually or with an actuation mechanism so that it can contact the patient's head (not shown). As the inner shell's inner surface 201A conforms to the patient's head, the sensors will contact the skull and be held in place by the rigid, semi-rigid, or semi-flexible inner shell 201.

Various specific embodiments may be envisioned for the invention. Examples presented are meant to provide illustration of the invention and its use and should not limit expression of the invention as presented herein.

### EXAMPLES

### Example 1

As shown in FIG. 4, an example a two-shell MEG helmet 200 was constructed with a rigid inner shell and a flexible cap as the outer shell. The rigid inner shell 201 was modeled after a patient's head shape, obtained using 3D scanning technology, and designed using computer aided design (CAD), and then finally 3D printed.

The 3D Systems' Sense 2 scanner was used to collect three-dimensional spatial data of the patient's head shape. This scanner uses highly sensitive infrared projector depth sensing technology to generate a complete polygon mesh model of the scanned object using their software called Unity Sense. Other available 3D scanners include XYZ Printing's 3D Scanner Pro, which uses depth sensing cameras and the more advanced Faro Arm scanner, which uses laser line probe in addition to depth sensing camera, or other 3D scanning technology may be utilized. Alternately, a mold of the patient's head may be made.

From the complete polygon mesh model of the patient's head, the inner shell 201 was modeled using CAD with Autodesk's Fusion 360 software. Other available CAD software include Dassault Systeme's Solidworks, PTC Creo, Autodesk Inventor, Catia v5 and Siemens NX. The patient's head polygon mesh model was imported into Fusion 360 and the inner shell was modeled such that its inner surface becomes an exact negative to the head's outer surface with a gap of 2 mm to allow for fitting over the patient's head. Other features such as mating features with the outer shell (not shown), bracket and sensor holders as well as vent holes (not shown) were modeled inside Fusion 360 and added to the model as well.

Once the inner shell 3D model file was completed, the “.stl” file was exported to a 3D printer. Other file types can also be exported for other 3D printers, including “.obj”, “.stp” and “.igs” files etc. For printing, a Raise3D N2 Plus 3D Printer was used. The Raise3D N2 Plus is a fused deposition modeling (FDM) 3D printer. FDM 3D printers are also called fused filament fabrication (FFF) 3D printers. In FDM and/or FFF printing, a continuous filament of a thermoplastic material is heated to a temperature of about 205° C. so that it can be extruded as a hair-thin filament and fused into the shape of the printed object. In our case, we used ABS plastic filament of 1.75 mm diameter to print the inner shell.

The interior surface 201A of the inner shell 201 was printed and fitted onto a patient's head. Since the interior surface 201A of inner shell 201 was an exact fit to the patient's head, any head movement is also directly translated to the inner shell, and by extension, all of the two-shell MEG helmet. To complete the two-shell MEG helmet, an outer shell 202 was made of a flexible cap of fabric fitted with ABS plastic sensor holders, modeled in similar fashion as the inner shell described previously in Fusion 360. The outer shell was made of a knit fabric being 90% polyester and 10% spandex to provide flexibility. The brackets 209 and sensor holders 210 were bolted onto it using plastic bolt features. The outer shell maintains the position of the sensors, allowing them to slide and lock onto the inner shell through the outer shell openings 204B into the inner shell openings 204A. Electronics in the form of flexible circuits (not shown) are integrated over the flexible outer shell and provides the necessary electrical connections to the sensors. The outer shell flexible cap was fitted over the inner shell on the patient's head to form the two-shell MEG helmet. The outer shell was destined to be able to fit over an inner shell of any size or relatively oval head shapes and can be removed and placed onto another patient's inner shell, thereby transferring all sensors with it. This offers the feature of giving the convenience of not having to remove and re-install each sensor individually from one patient to the next.

### Example 2

As illustrated in FIG. 5, Another example two-shell MEG helmet with hard outer shell 202. The inner shell was modeled to fit the patient's head shape using a mold, then 3D scanned and 3D printed as described in Example 1. This process provides an inner shell which fits exactly to the patient's head such that any head movement is translated to the inner shell and the rest of the two-shell MEG helmet to ensure sensor positions relative to the brain are fixed.

In this case, to make the inner shell, clay putty was used to cover the patient's head to form the mold of the inner shell's inner surface. Once the clay dried to achieve some rigidity, it was removed from the patient's head to dry fully. Using 3D Systems' Sense 2 scanner the physical mold was converted into a polygon mesh that was imported into Fusion 360, CAD software to complete the modeling of the inner shell. Other 3D scanners and CAD software available for use are described in Example 1. Other features including the mating features with the outer shell, bracket and sensor holder and vent holes were added to the inner shell using the Fusion 360 software. Once the inner shell model was complete it was sent to the Raise3D N2 Plus 3D printer as an .stl file for fabrication. Other available 3D printing software are mentioned in Example 1.

For this example, the outer shell 202 was made of a rigid material as is illustrated in FIG. 5. The outer shell was modeled using CAD to fit over the inner shell 201 and provides alignment for each sensor to slide into place on the inner shell as shown in FIG. 7. The outer shell was 3D printed using Raise3D N2 Plus with ABS plastic 1.75 mm diameter filament. Electronics in the form of wiring and connectors are soldered together and installed on the outer shell. They provide the necessary electrical connections to the sensors for data collection and sensor control. The hard, outer shell can be removed and installed from one patient to another, along with all the attached individual sensors. This saves time and energy for setting up the sensors in order to perform scans and data collection.

Although the present invention has been described with reference to the disclosed embodiments, numerous modifications and variations can be made and still the result will come within the scope of the invention. No limitation with respect to the specific embodiments disclosed herein is intended or should be inferred. Each apparatus embodiment described herein has numerous equivalents.

What is claimed is:

1. A multi-shell customizable magnetoencephalography (MEG) helmet designed to cover a scalp of a human and housing at least two magnetic field sensors at known locations with respect to the helmet geometry comprising:
a) at least one wearable custom-made inner shell designed to substantially cover a scalp having an interior surface designed to match the outer shape and size of the scalp and an exterior surface designed to mate with a reusable, pliable, and wearable outer shell;
b) at least two housings in the inner shell and the outer shell designed to receive at least two magnetic field sensors at known locations with respect to the helmet geometry; and
c) wherein any number of inner shells can be substituted without removing the sensors housed on the outer shell.

2. The multi-shell customizable magnetoencephalography (MEG) helmet of claim 1, wherein the inner shell further comprises openings extending through the inner shell designed to hold the two or more magnetic sensors, and wherein the openings are configured to align with the sensor housing structures of the outer shell.

3. The multi-shell customizable magnetoencephalography (MEG) helmet of claim 1, wherein the sensor housings of the outer shell are openings extending through the outer shell.

4. The multi-shell customizable magnetoencephalography (MEG) helmet of claim 1, wherein the sensor housing structures are brackets.

5. The multi-shell customizable magnetoencephalography (MEG) helmet of claim 1, wherein the inner shell is rigid.

6. The multi-shell customizable magnetoencephalography (MEG) helmet of claim 1, wherein inner shell is semi-rigid.

7. The multi-shell customizable magnetoencephalography (MEG) helmet of claim 1, wherein the inner shell is manufactured using three-dimensional (3D) printing.

8. (canceled)

9. The multi-shell customizable magnetoencephalography (MEG) helmet of claim 1, wherein the outer shell is made of a rigid material.

10. (canceled)

11. (canceled)

12. (canceled)

13. (canceled)

14. (canceled)

15. (canceled)

16. A method of constructing a magnetoencephalography helmet, the method comprising
the steps of:
a) forming a custom inner shell with inner surface that conforms to a patient's head;
b) forming a generic outer shell that fits over any number of custom inner shells; and
c) fitting sensors to the outer shell.

17. The method of claim 16 further comprising including openings in the outer shell to
house the sensors.

18. The method of claim 16 further comprising including openings in the inner shell to
house the sensors.

19. The method of claim 16 further comprising providing an opening for the entire face of the patient.

20. A method of creating a magnetic emissions image of the brain, the method comprising the steps of:
a) forming a custom inner shell with inner surface that conforms to an individual patient's head;
b) forming a reusable and pliable outer shell that fits over any number of custom inner shells;
c) fitting sensors and associated communications hardware on the outer shell;
d) fitting the outer shell over the custom inner shell and moving sensors to the shell prior to f;
e) fitting the custom inner shell onto the patient's head;
f) following e, operating the sensors to collect data pertaining to magnetic waves produced by the patient's head; and
g) following f, moving sensors to the outer shell to replace the inner shell without removing the sensors and associated communications hardware from the helmet.

21. A multi-shell customizable magnetoencephalography (MEG) helmet comprising:
a) a custom-made inner shell designed to fit an individual patient's head having an interior surface designed to match a shape and size of the form of a substantial portion a patient's scalp;
b) a pliable outer shell comprising at least two housings to receive at least two magnetometers;
c) at least two housings on the inner shell designed to align with the at least two housings on the outer shell such that the at least two magnetometers on the outer shell can slide into the at least two housings on the inner shell;
d) wherein an exterior surface of the inner shell is designed to mate with an interior surface of the outer shell such that the inner and outer shells do not move in relation to one another when in use; and
e) wherein in any number of inner shells can be substituted without removing the sensors from the outer shell.

22. The multi-shell customizable magnetoencephalography (MEG) helmet of claim 21, wherein the outer shell further comprises cables for carrying signals from the magnetometers, and are on the outer shell such that the inner shell can be replaced without any physical interaction with the cables.

23. The multi-shell customizable magnetoencephalography (MEG) helmet of claim 21, wherein the interior surface of the custom-made inner shell is designed to leave at least a 2 mm gap between the interior surface and the scalp of the patient's head.

24. The multi-shell customizable magnetoencephalography (MEG) helmet of claim 21, wherein the inner shell is rigid.

25. The multi-shell customizable magnetoencephalography (MEG) helmet of claim 1, wherein the outer shell further comprises cables for carrying signals from the sensors, and are on the outer shell such that the inner shell can be replaced without any physical interaction with the cables.

26. The multi-shell customizable magnetoencephalography (MEG) helmet of claim 1, wherein the interior surface of the custom-made inner shell is designed to leave at least a 2 mm gap between the interior surface and the scalp of the patient's head.

27. The multi-shell customizable magnetoencephalography (MEG) helmet of claim 1, wherein the inner shell is rigid.
